(19) United States
(12) Patent Application Publication
(10) Pub. No.: US 2024/0285351 A1
(43) Pub. Date: Aug. 29, 2024

(54) SURGICAL ASSISTANCE SYSTEM WITH IMPROVED REGISTRATION, AND REGISTRATION METHOD

(71) Applicant: B. Braun New Ventures GmbH, Freiburg im Breisgau (DE)

(72) Inventors: Jean Stawiaski, Kirchzarten (DE); Maximilian Dippel, Freiburg (DE); Amir Sarvestani, Freiburg (DE)

(21) Appl. No.: 18/575,221

(22) PCT Filed: Jun. 29, 2022
(86) PCT No.: PCT/EP2022/067925

§ 371 Date: Dec. 28, 2023

(30) Foreign Application Priority Data
Jul. 1, 2021 (DE) 10 2021 117 004.4

(51) Int. Cl. A61B 34/20; G06T 7/37

(52) U.S. Cl. CPC A61B 34/20; G06T 7/37; A61B 2034/2065

(57) ABSTRACT

A surgical assistance system, registration method and computer-readable storage are used in a surgical intervention. The system includes a 3D recording device that generates a three-dimensional intracorporeal recording of a patient; a tracking system that detects and tracks a surgical intervention region of the patient; a data unit that generates recorded 3D data; and a control unit that processes the intracorporeal recording, data of the tracking system, and the 3D data. The system registers the 3D data onto an outer surface of the patient as a first registration. The control unit determines a landmark and/or a surface and/or a three-dimensional volume of an intracorporeal structure as an intracorporeal reference from the intracorporeal recording and/or the 3D data and registers the 3D data on the intracorporeal structure of the patient detected as an intracorporeal recording as a second registration based on the first registration and the intracorporeal reference.

### CROSS-REFERENCE TO RELATED APPLICATIONS

This application is the United States national stage entry of International Application No. PCT/EP2022/067925, filed on Jun. 29, 2022, and claims priority to German Application No. 10 2021 117 004.4, filed on Jul. 1, 2021. The contents of International Application No. PCT/EP2022/067925 and German Application No. 10 2021 117 004.4 are incorporated by reference herein in their entireties.

### FIELD

The present disclosure relates to a surgical assistance system for use in a surgical intervention, comprising: at least one, in particular medical, imaging 3D capturing device/3D imaging device, which is provided and adapted to create a three-dimensional, in particular magnified, intracorporeal image for a patient and to provide it in a computer-readable manner; a tracking system/monitoring system, in particular a surgical navigation system, which is provided and adapted to detect and track a surgical intervention region of the patient, in particular the patient with an outer surface/external structure, preferably together with at least a portion of the 3D capturing device; a data provision unit, in particular a storage unit, which is adapted to provide digital 3D image data, in particular preoperative 3D image data, of the patient; and a control unit adapted to process the three-dimensional intracorporeal image, the data of the tracking system, and the provided 3D image data. In addition, the disclosure relates to a registering method/register method/registration method and a computer-readable storage medium.

### BACKGROUND

Surgical navigation systems are a standard in neurosurgery today. They can be used to navigate an intervention and guide an instrument to reach a surgical target site. Although navigation systems enable the surgeon to identify anatomical structures during surgery, their accuracy is often strongly limited in a disadvantageous way. The accuracy is decisively defined or specified by the registration between digital (3D) image data to/on the patient, i.e. in particular the assignment of a reference image/reference picture from preoperative 3D image data (e.g. MRI image data or CT image data) to the intraoperative, i.e. body internal, reference image of the patient. In this way, the virtual structures are linked and correlated with the real structures.

Such registration is usually performed by detecting points on an outer surface of the patient (e.g. points on the patient's face) as reference points and then assigning these reference points to the corresponding points in the preoperative 3D image data. Various registration methods are available, in particular point-to-point matching or surface matching between the 3D image data and the patient. The reference points to be measured can be detected by palpation or without contact using sensors such as video cameras or lasers. When using intraoperative 3D imaging via a three-dimensional (3D) capturing device, registration can also be carried out by using so-called ‘fiducials’ (optical reference points) on the patient, which become visible in the 3D image data/in the 3D image set and enable automatic registration.

All these registration methods have in common that they use outer surfaces of the patient (i.e. external, visible surfaces of the human body, such as skin, a face, a skin section with characteristic features such as moles), and that the registration is performed before the start of surgery. After opening the body for an intracorporeal intervention and during surgery, tissue is almost always moved, whether intentionally and due to a resection or unintentionally and due to collapsing or deflating tissue. In particular in the region of a neurosurgical opening of a patient's skull during craniotomy, the collapsing tissue is known as brain displacement caused by the craniotomy or by removal of tissue.

However, due to tissue displacement, this results in disadvantageous inaccuracies between the real tissue and the tissue in the 3D image data of one centimeter or even more. These deviations make precise localization of the tissue by navigation impossible and carry a high risk of unintentional dangerous injury to the tissue due to incorrect navigation.

In order to overcome this limitation of the deviation between the moving tissue and the 3D image data, there are, for example, solutions that use computer tomography (CT) or magnetic resonance imaging (MRI) during surgery, i.e. intraoperatively, to capture and provide new 3D image data during the surgery itself. However, such solutions significantly increase the surgery duration and cause the costs per intervention to rise sharply. It may also be the case that, due to the already long surgery duration, an intervention becomes impossible or can only be performed on several day with a time interruption.

An alternative approach for determining a deviation and adjusting the 3D image data to the actual pose of the tissue is the use of intraoperative ultrasound (during surgery). With ultrasound, a loss of accuracy can be at least partially corrected. However, ultrasound images have the disadvantage that they are difficult to interpret and do not provide the required high accuracy. In the craniotomy field in particular, navigation must be performed with the highest precision. In addition, the use of an ultrasound device also increases the duration and complexity of surgery. An ultrasound device also requires a standing area in the already quite cramped operating room and reduces accessibility to the surgical region.

Another solution provides a method for compensating for a brain shift, for example, by using the microscope image/the microscope picture and well-defined anatomical structures (in particular vessels) to manually shift the 3D image data/3D picture data in such a way that it ultimately matches the microscope image at least roughly. However, this method is very subjective and requires manual adjustment, which further delays an intervention. In addition, this method does not take into account all six possible degrees of freedom (three degrees of freedom of a translation and three degrees of freedom of a rotation), as only a translational correction is performed.

For example, U.S. Pat. No. 9,336,592 B2 discloses a system based on matching 3D surfaces extracted from preoperative 3D image data/3D data set with a (real) 3D surface extracted from a stereo camera system.

### SUMMARY

It is therefore the object of the present disclosure to avoid or at least reduce the disadvantages of the prior art and, in particular, to provide a surgical assistance system, a registration method and a computer-readable storage medium with which a registration is further improved and an accuracy of the registration is increased. A fundamental object can be seen in particular in maintaining a high accuracy of a navigation during the entire intervention, in particular when tissue is spatially changed and moved by the intervention. Another partial object can be seen in performing simple intraoperative registration without the need for a biomechanical model. Furthermore, another partial object can be seen in the ability to perform a new registration intraoperatively (i.e. during the intervention) at any time in order to increase accuracy as required and, above all, to provide fast registration. A further partial object can also be seen in minimizing the computational effort required for registration.

In principle, in the present disclosure, a two-step registration is proposed for the surgical assistance system and also for the registering method/registration method, in particular in order to simplify and reduce the computational effort of the registration and to limit a range of errors. A basic idea of the present disclosure is that the assistance system or the registering method is adapted to perform the registration in at least two steps (hereinafter referred to as first (registration) step A and second (registration) step B). While in the first registration step A, an outer surface or structure of the patient, in particular a face, is used for an initial registration, in the second registration step B, an internal/intracorporeal structure or tissue of the patient is used for a refined and a chronologically subsequent registration. The first registration step A leads to a (standardized) first registration, which is initially still quite accurate before the intervention. This first registration step A or the first registration is carried out in particular before the start of the intervention, i.e. before the patient is opened and the intervention begins (for example in the case of an intervention with a skull opening, a dura opening, a tissue resection or a tumor removal).

After opening the patient and after the intervention has begun and the manipulation of the tissue with the accompanying pose change of the tissue affects the accuracy of the registration, the second registration step B is performed to adapt the registration to the changed tissue pose and to refine it even further, so to speak. The registration from registration step A is used as the basis. The second registration step B to be adapted can be performed at any time during the intervention, usually when a critical resection begins (for example, a resection of tumor borders) and when the surgeon requires high precision to localize the position of the resection instrument.

The registration of the second registration step B is based on an intraoperative visualization system/a 3D capturing device that creates three-dimensional (3D) intracorporeal images and enables the surgeon to localize and preferably also place identifiable anatomical landmarks of the operated soft tissue within the surgical region. These landmarks may be, for example, sulcus structures, vessels or lesions. The surgeon can use the visualization system/3D capturing device in order to detect selected landmarks that correspond to the landmarks identified in the 3D image data, in particular the preoperative 3D image data, or vice versa. In particular, the 3D capturing device may have a stereo camera (i.e. two optical systems at a distance from each other) in order to calculate and create a three-dimensional intracorporeal image from the two offset (two-dimensional) intracorporeal images or respectively from two different viewing positions.

In other words, in particular a surgical assistance system is proposed comprising a tracking system, in particular a surgical navigation system, and a 3D visualization system (3D capturing system with 3D capturing device), wherein the 3D visualization system is adapted to generate three-dimensional, i.e. spatial, intracorporeal images of internal structures of the patient, and the tracking system is adapted to follow/track the 3D visualization system. The system is adapted to register 3D image data, which were captured in particular before the intervention (i.e. preoperatively), on this patient using an outer surface of the patient (outer surface). The 3D visualization system (3D capturing system with 3D capturing device) generates intracorporeal three-dimensional images/3D images with at least one three-dimensional position (3D position with three coordinates) and/or at least one pose of intracorporeal structures from the interior or internal tissue of the patient (in the vicinity of the surgery region). These internal structures from the three-dimensional intracorporeal image with the at least one three-dimensional position are correlated with corresponding (intracorporeal) structures from the 3D image data, in particular from preoperative 3D image data/3D image set, in order to improve the registration and to increase the accuracy of the registration for the region of interest.

An advantage is that this assistance system and also the registering method allows a correction of accuracy errors resulting from the surgical workflow (for example a slight displacement of a tracker/marker during draping) and is not limited to a correction of a tissue displacement, in particular a brain displacement. Furthermore, the surgical assistance system can be used continuously during the intervention in order to increase the accuracy of a registration.

A further advantage is that not only intraoperative, two-dimensional images with reduced information content (2D data) are used, which would require a large exposure of the brain and a biomechanical model of the brain, but that only a small local intervention region is required due to the three-dimensional, intracorporeal image, without the need for a biomechanical model.

In yet other words, a surgical assistance system and registration method is proposed that ensures accurate registration during the use of surgical navigation by performing a two-stage registration process with initial registration before the intervention using the patient's outer surface and with refined registration after the start of the intervention using the patient's internal structure in the vicinity of the surgery region.

In yet further words, the surgical assistance system, in particular the control unit, is adapted to register the 3D image data on an outer surface of the patient as a first registration and, in particular, to store this first registration in the storage unit; and the control unit is adapted to determine at least a landmark and/or a surface and/or a three-dimensional volume of an intracorporeal tissue as an intracorporeal reference from/in the three-dimensional intracorporeal image and/or the 3D image data, and on the basis of the first registration and on the basis of the determined intracorporeal reference, to register/correlate the 3D image data to the (inner) intracorporeal tissue/structure of the three-dimensional intracorporeal image (IA) as a second registration in order to increase the accuracy of the registration. Based on the existing basis of the first registration, a correlation can be carried out particularly quickly and efficiently in the second registration. This also prevents or at least minimizes possible errors of incorrect registration that may occur due to the calculation.

The term ‘3D image data’ defines three-dimensional, spatial image data, i.e. with three dimensions, for example in an X, Y and Z direction.

The term ‘outer surface’ refers to a body surface of the patient that is normally visible without an opening or incision. In particular, the outer surface is the patient's skin. In other words, an externally visible structure of the patient is detected.

The term ‘position’ refers to a geometric position in three-dimensional space, which is specified in particular via coordinates of a Cartesian coordinate system. In particular, the position can be specified by the three coordinates X, Y and Z.

The term ‘orientation’ in turn indicates an orientation (such as position) in space. It can also be said that the orientation indicates a direction or rotation in three-dimensional space. In particular, the orientation can be specified using three angles.

The term ‘pose’ includes both a position and an orientation. In particular, the pose can be specified using six coordinates, three position coordinates X, Y and Z and three angular coordinates for the orientation.

Advantageous embodiments are explained in particular below.

According to a preferred embodiment, the control unit may be adapted to perform the second registration on the basis of a rigid transformation. In this context, a rigid transformation means that the three-dimensional intracorporeal image is only translationally shifted and/or rotationally rotated in relation to the 3D image data. This is beneficial for a necessary computational effort. The 3D image data is therefore not adapted in such a way that a local distortion or deformation is taken into account. The registration correction performed is therefore a rigid transformation that does not take into account any elastic deformation of the brain. This rigid transformation hardly affects the surgical accuracy, since the registration correction is performed precisely in the region of the surgery, so that the rigid transformation provides the required precision in this region of interest, while outside this region the precision is insignificant in the case of elastic deformation. One advantage of this is that no biomechanical model is needed for adaptation. In other words, the correlated landmarks and/or surfaces can be adapted, in particular using a rigid transformation, in order to refine the registration and to obtain high navigation accuracy in the region of interest.

According to a preferred embodiment, the surgical assistance system may comprise a surgical 3D microscope and/or a surgical 3D endoscope and/or a medical instrument with an optical 3D camera as 3D capturing device for creating the three-dimensional intracorporeal image. All three medical devices, i.e. the surgical (3D) microscope, the surgical (3D) endoscope and the instrument with the 3D camera are adapted to create three-dimensional intracorporeal images from inside the patient's body. In other words, the intraoperative 3D capturing device (the intraoperative imaging device) may be a 3D microscope, a 3D endoscope or an optical camera system that generates the (magnified) image from inside the patient, in particular in the form of 3D point cloud data (i.e. a set of points in three-dimensional space for geomorphologic analyses, for example).

According to an aspect of the present disclosure, the control unit may be adapted to determine at least one point-like landmark and/or at least one surface for the second registration/correlation in the three-dimensional intracorporeal image. Alternatively or in addition to landmarks, surfaces may also be used. Both surfaces and landmarks can be automatically extracted and matched using standard image processing algorithms. For this purpose, corresponding surfaces have to be selected between the 3D image data and the intraoperative image of the visualization system, wherein the surgical assistance system allows manual input via a touch display, and/or the control unit is adapted to perform the correlation or registration automatically. In the intracorporeal structure of the patient's internal tissues, punctiform orientation points and/or surfaces can therefore be determined.

According to a further aspect of the disclosure, the control unit may be adapted to detect or determine exactly one single landmark, in particular a single landmark point in space, and to perform the registration, in particular the second registration, on the basis of the one landmark, in particular the landmark point, with only one transformation correction with respect to a translation, while a rotation/turn is not changed/remains unchanged. This configuration represents the simplest and, from a computational point of view, the most efficient and fastest correction of the registration. In particular, the second registration can be carried out with just a single set landmark point. With one landmark, the registration offset can be corrected in at least three degrees of freedom (translation only). In particular, only one landmark can be used to perform a transformation correction while the rotation remains unchanged.

According to a further aspect of the disclosure, the control unit may be adapted to detect or determine exactly two landmarks, in particular two spaced landmark points, in space and to perform the registration on the basis of the two landmarks. The two landmarks can be used to correct 5 degrees of freedom (translation and two rotations).

In particular, the control unit may be adapted to detect or determine at least three landmarks, in particular at least three spaced landmark points, in space and perform registration on the basis of the at least three landmarks. With three or more landmarks, all six degrees of freedom can be corrected (translation and three rotations).

According to a preferred embodiment, the surgical assistance system may comprise a 3D surgical microscope, wherein the focal point of the microscope is detected and processed by the control unit and the landmark (position) is detected intraoperatively based on the focal point. The control unit is particularly adapted to track the microscope via the tracking system, in particular the navigation system, and to determine a position and/or orientation of a microscope head with optical system in order to determine the position of the focal point relative to the patient or to the patient reference system.

According to a further preferred embodiment, the surgical assistance system may comprise a surgical tool and may be adapted to geometrically detect the surgical tool, in particular a suction hose, and to track it by the tracking system, and furthermore the control unit may be adapted to determine a position of a distal tip, in particular the distal tip of the suction hose, upon a command input by the user and to set the landmark based on this position. A user can therefore preferably simply point the distal end of the suction hose at the desired landmark and, for example, perform a command input to set a landmark on a touch display in order to detect this landmark. In particular, the three-dimensional intracorporeal image of the 3D capturing device (the intraoperative visualization device) can be used for detection, since the position of the landmark reflects the real position.

Preferably, the tracking system, in particular the navigation system, may comprise an infrared-based navigation system and/or an electromagnetic navigation system and/or an image-processing navigation system. In the case of an infrared-based navigation system, infrared markers/infrared trackers are preferably provided on the patient and on the 3D capturing device. Optionally, further infrared markers may be provided at the provided (either still closed or opened) surgical intervention region in order to precisely detect this region of particular interest and, for example, to perform an initial registration based on this region. In an electromagnetic navigation system (EM-navigation system), EM sensors may be attached to the patient and/or 3D capturing device. In an image-processing navigation system, navigation is performed based on image analysis using machine vision to detect movement such as translation and rotation. The tracking system, in particular the surgical navigation system, may therefore be based on infrared tracking/infrared-based tracking, EM tracking/electromagnetic tracking and/or machine vision tracking/optical image processing tracking.

According to a preferred embodiment, the control unit may be adapted to perform the first registration via a point-to-point matching and/or a surface matching using the outer surface/structure of the patient. The first registration or the registration method of the first registration step A may thus be based in particular on a point-to-point registration and/or a surface matching and/or a video registration and/or a fiducial matching/mapping using an intraoperative 3D scanner such as a computer tomograph (CT) or a magnetic resonance tomograph (MRT). The first registration with preferably point-to-point matching and/or surface matching is performed using the external structure or the outer surface of the patient.

The objects are solved with respect to the registering method/registration method for registering 3D image data on a tissue of a patient, in particular for a surgical assistance system according to the present disclosure, by the steps of: detecting 3D image data of a patient; registering the 3D image data onto an outer surface of the patient as a first registration; creating a three-dimensional intracorporeal image of the patient by a 3D capturing device; determining at least a landmark and/or a surface and/or a volume in the three-dimensional intracorporeal image and/or in the 3D image data as an intracorporeal reference; and registering, based on the first registration and the determined intracorporeal reference, the 3D image data to the internal structures captured by the three-dimensional intracorporeal image as a second registration in order to increase an accuracy of the registration. Through this registration method.

According to a preferred embodiment, the registering method may further comprise the steps of: detecting at least a landmark and/or a surface in the three-dimensional intracorporeal image; and performing a rigid transformation in order to register the 3D image data to the intracorporeal structure. This further reduces a computational effort by, for example, a processor, since the rigid transformation does not require complex mathematical calculations.

The objects of the present disclosure are solved with respect to a computer-readable storage medium, in that it comprises commands which, when executed by a computer, cause the computer to execute the method steps of the registering method according to the present disclosure.

Any disclosure related to the surgical assistance system of the present disclosure also applies to the registering method of the present disclosure, just as any disclosure related to the registering method according to the present disclosure also applies to the surgical assistance system of the present disclosure. In particular, the features of the adaptation of the control unit can be used analogously for a method step as well as a method step for a corresponding analogous adaptation of the control unit.

### BRIEF DESCRIPTION OF THE DRAWINGS

The invention is explained below with reference to preferred embodiments with the aid of the accompanying Figures.

FIG. 1 shows a schematic, perspective view of a surgical assistance system according to a first preferred embodiment,

FIG. 2 shows a schematic view of a registering method/registration method of a preferred embodiment,

FIG. 3 shows a schematic representation for explaining a registering method with two-stage registration, and

FIG. 4 shows a flow diagram of a registering method of a further preferred embodiment.

The Figures are schematic in nature and are only intended to aid understanding of the invention. Identical elements are marked with the same reference signs. The features of the various embodiments can be interchanged.

### DETAILED DESCRIPTION

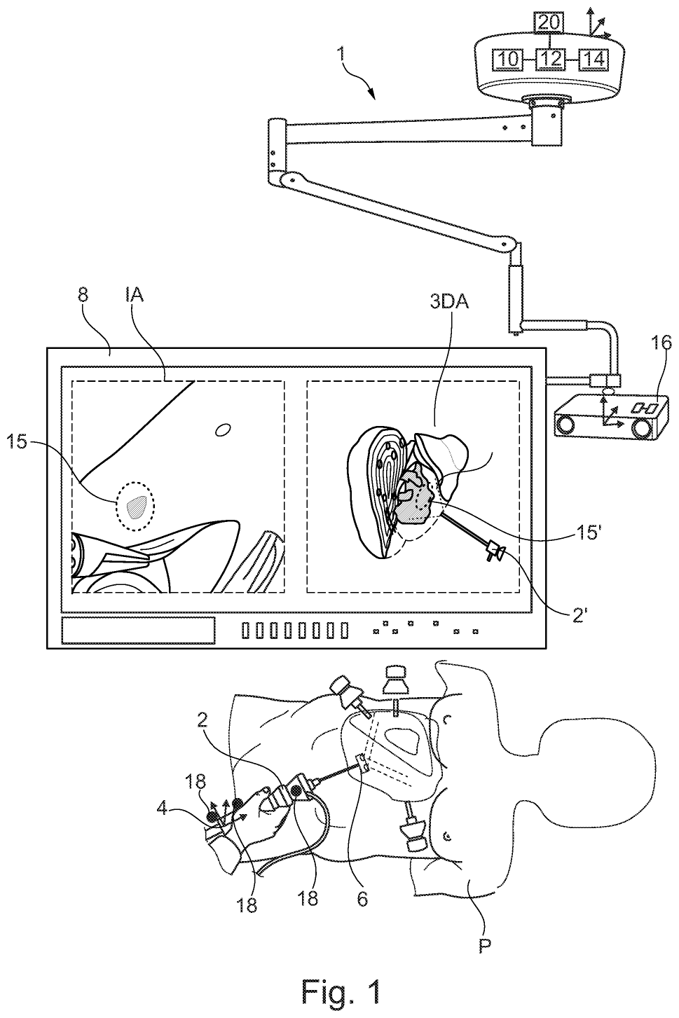

FIG. 1 shows a schematic view of a surgical assistance system 1 (hereinafter only referred to as assistance system) of a first preferred embodiment. The assistance system 1 is used in a medical sterile room. An open intervention is performed on the patient in a central, sterile surgical intervention region. A rigid 3D video endoscope 2 (hereinafter referred to as endoscope) with a handpiece/handle 4 and a spaced, front-side imaging 3D image head 6 is positioned on the endoscope shaft side inside the patient's body. For imaging, the 3D image head 6 has a 3D endoscope video camera in the form of a distal stereo camera, which creates a three-dimensional intracorporeal image IA of the inside of the patient's body P as a live video image at the front-side in the direction of a longitudinal axis of the endoscope 2 and provides it digitally. A display device in the form of a surgical monitor 8 can then be used to display visual content for those involved in the operation for navigation. It should be noted that the endoscope 2 can alternatively also be guided by a robotic arm at its handling portion.

Furthermore, the surgical assistance system 1 has a data provision unit 14 in the form of a storage, in which digital, preoperative 3D image data 3DA of the patient P to be treated is stored in a digital/computer-readable manner. For example, (segmented) MRI image data or CT image data can be stored as 3D image data 3DA, which virtually or digitally depict a body of the patient P or at least a part of the body.

For detection, processing, calculation and control, the assistance system 1 has a control unit 12 which is adapted to process the three-dimensional intracorporeal image IA of the endoscope 2 and the 3D image data 3DA.

The surgical assistance system 1 furthermore has a tracking system 14 which, among other things, continuously detects movement of the endoscope 2 and the image head 6 and provides the control unit 12 with endoscope pose data. In the present embodiment, the tracking system 14 has an infrared-based navigation system with an external 3D stereo camera 16 and infrared markers 18, which are provided on the handle. Alternatively or additionally, a combination with an endoscope-internal sensor, such as an IMU sensor, and/or with an image analysis performed by the control unit 12 can also be used.

In contrast to the prior art, the surgical assistance system 1 according to this embodiment is adapted to perform a two-stage registration with an external patient structure for the first registration and then to use the internal structure of the patient P for an intraoperative, second registration for a refined registration on the basis of this first registration and on the basis of captured landmarks via the 3D capturing device in the form of the endoscope 2.

Specifically, the tracking system 14 and the control unit 12 are adapted to first register the 3D image data 3DA via the tracking system 14 with the external 3D stereo camera 16 on an outer surface of the patient P, in this case the human body with the (planned) intervention region and the head as the first registration. This first registration usually takes place before the intervention, when patient P has been placed in the surgery room. The transformation matrix determined in the first registration is stored in the storage unit 20. This completes the initial or first registration as registration step A and provides the surgical assistance system with an overview registration or basic registration for the entire surgery.

Furthermore, the control unit 12 is adapted to detect anatomical landmarks 15 in the three-dimensional intracorporeal image IA of the endoscope 2 after the start of the intervention and to determine corresponding anatomical landmarks 15′ in the 3D image data provided. In the three-dimensional intracorporeal image IA of the endoscope 2, which is inserted into the body of the patient P and captures a targeted anatomical structure as a three-dimensional image, the surgeon selects three predefined characteristic (real) landmarks 15 in this embodiment. These three landmarks 15 in real space can also be found as three (virtual) landmarks 15′ in virtual space, just as the endoscope 2 can be displayed in virtual space as a virtual endoscope 2′. The three-dimensional intracorporeal image IA is correlated, i.e. registered, with the 3D image data via the two times three corresponding anatomical landmarks 15, 15′.

The control unit 12 is specially adapted to determine three landmarks and/or a surface of the inner structure or an intracorporeal tissue as an intracorporeal reference from the three-dimensional intracorporeal image IA. Then, based on the first registration and the determined intracorporeal reference, the control unit 12 registers the 3D image data 3DA to the (inner) intracorporeal tissue/the inner structure as a second registration in order to increase the accuracy of the registration. For example, if the liver is moved, the surgeon can use the surgical assistance system 1 to perform the second registration again and re-register to the actual pose of the liver in the intervention region of interest.

The surgical assistance system 1 is therefore specially adapted to perform a two-stage registration method, wherein the second registration can be repeated intraoperatively several times at any desired time.

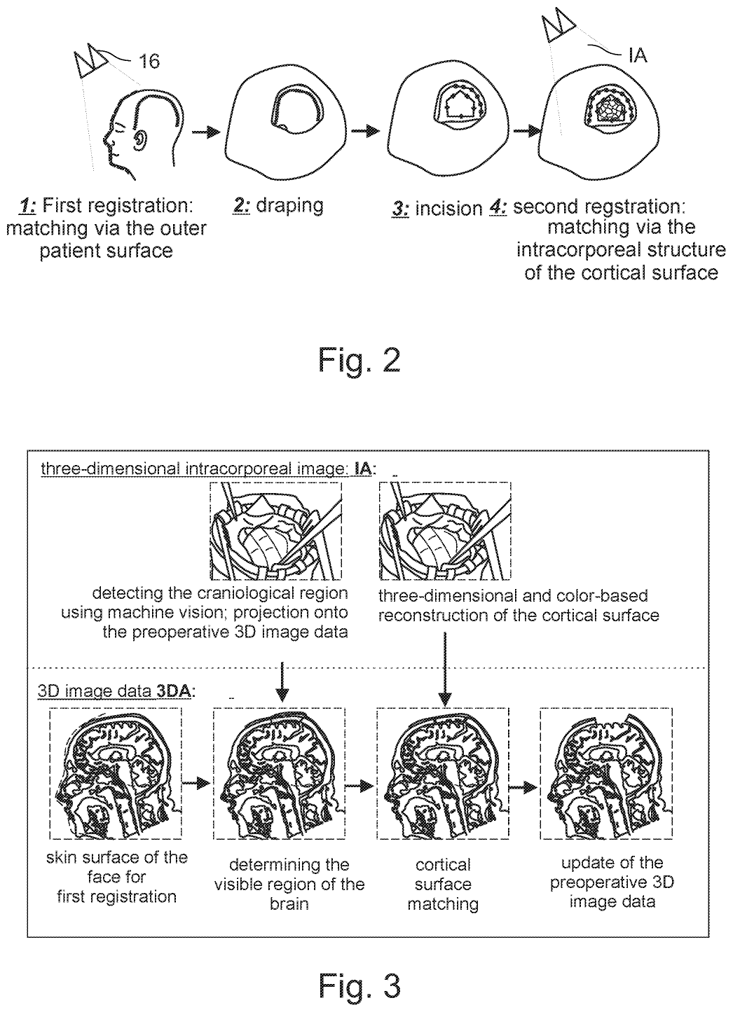

FIG. 2 shows a schematic view of a registering method for registering 3D image data 3DA on a tissue of a patient P according to a first preferred embodiment. First, a first registration of the 3D image data 3DA of the patient P onto the front surface of the patient's face is performed. The geometric structure of the 3D image data of the patient's face is correlated with the captured three-dimensional structure of the real face of the patient P in order to obtain a first transformation between the 3D image data and the patient P. According to this embodiment, this step is performed before an intervention. After the first registration is completed as an overview registration, the skull of patient P can be draped around the planned intervention site.

This is followed by an incision step in which the skull is opened and the inner structure of the cerebrum is then visible. Due to the incision, the opening and the manipulation with medical instruments, the pose of the brain can change in some areas, so that the 3D image data 3DA in the region of the target intervention no longer corresponds to the actual internal structure of the patient P. Therefore, a second registration is performed. A second registration is therefore performed. Here, the visible intracorporeal structure of the cortical surface is spatially detected by the 3D capturing device and is compared with the 3D image data 3DA via suitable landmarks and/or surfaces, is correlated and thus registered as a second registration. In particular, a transformation matrix is determined that performs a transformation only within defined limits compared to the first registration with first transformation, in particular transformation matrix.

The first registration therefore defines the basis, and the second registration is only carried out in a defined region (a predefined tolerance range) compared to the first registration in order to refine the registration intraoperatively at any time. Based on the first registration, a correlation is performed particularly quickly and efficiently. Possible errors of incorrect registration, which may occur due to the calculation, are also minimized. For example, if only a small structure of 3D image data 3DA of the body of the patient P is available, such as a muscle section in the left arm, only the muscle section in the left arm and not in the right arm can be determined via the correlation. In other words, the two-stage method with initial registration provides a rough minimum accuracy with the first registration, which can be refined later but cannot be made any coarser.

FIG. 3 shows a schematic sequence of a registering method according to a further preferred embodiment. This registering method is characterized by the fact that on the basis of the three-dimensional intracorporeal image IA, even the 3D image data 3DA are adapted to the actual structure after opening the skull.

First, the (three-dimensional structure) of the skin surface of the face of the patient P in the 3D image data 3DA as well as in real life is used for an initial registration.

After opening the skull, a craniological or cerebral region is spatially recognized or detected by machine vision in the three-dimensional intracorporeal image IA and projected onto the preoperative 3D image data 3DA. In this way, a visible region of the brain can be determined in the 3D image data 3DA.

Furthermore, a three-dimensional and color-based reconstruction of the cortical surface is performed using three-dimensional intracorporeal image IA, which is also color-based.

This reconstruction is used for the 3D image data 3DA to perform a cortical surface matching.

In the next step, the preoperative 3D image data 3DA is then adapted so that the intervention region is updated via the three-dimensional reconstruction, i.e. the removed skullcap is removed from the 3D image data 3DA and the pose of the brain is adapted.

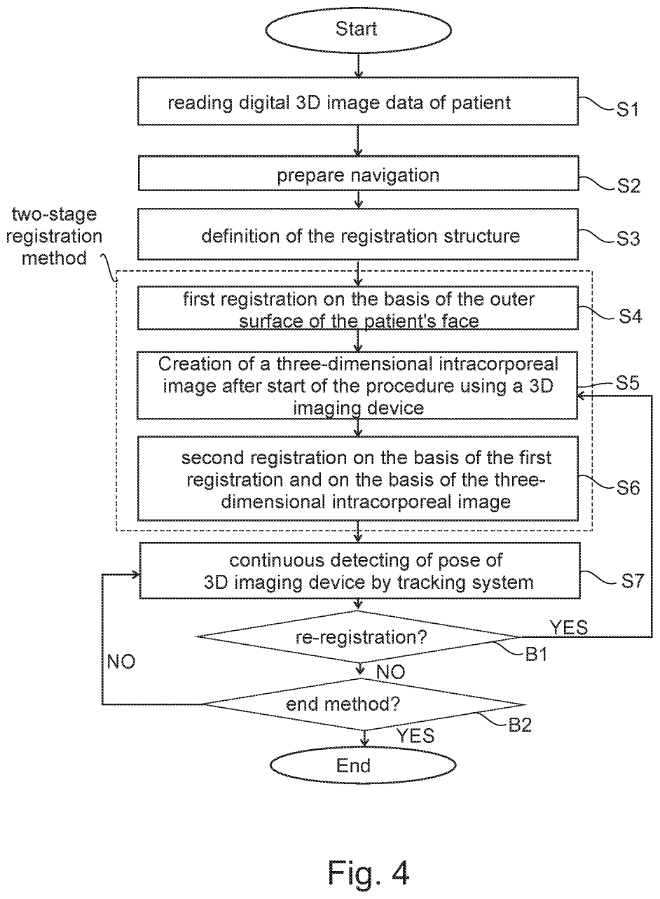

FIG. 4 shows a flow chart of a registering method of another preferred embodiment with a two-stage registration process.

In a first step S1, preoperative 3D image data 3DA is detected, in particular MRI image data or CT image data.

In a second step S2, the navigation is prepared or set up. In particular, trackers are provided on the patient, on the 3D capturing device (3D visualization system) and/or on an instrument.

In a subsequent step S3, the registering structure is preferably defined. Here, for example, landmarks and/or surfaces are determined in the preoperative 3D image data 3DA. For example, a three-dimensional structure of the patient's face can be defined as the registering structure for the first registration and/or a cerebral region for the second registration.

In the next step S4, the first registration takes place. This is based in particular on the previously defined outer surface of patient P's face.

The first registration can be based on point-to-point registration and/or surface matching (mapping) and/or video surface acquisition, such as a stereo camera, and/or fiducial mapping.

The first registration is then completed and the interventional region can be draped and opened by incision.

After starting the intervention, a 3D capturing device is used in step S5 to create a three-dimensional intracorporeal image IA, which is used to refine the registration. At least one landmark and/or surface is determined in the three-dimensional intracorporeal image IA, in particular the defined registering structure is used, and the intraoperative structure of the patient is matched with the preoperative structure of the patient for the second registration. In this step, in particular only a rigid transformation (i.e. only a displacement and a rotation of the three-dimensional images IA, 3DA relative to each other) is performed in order to increase the accuracy of the registration for the target area of interest.

The surgery can then be continued and a very high level of navigation precision can be achieved thanks to the registration refinement provided by the second registration. If instruments are now used for a surgery, they can be tracked via a tracking system and can be precisely tracked in the 3D image data 3DA, so that navigation is possible using only the 3D image data 3DA in order to reach the target application area with the instrument.

The pose of the 3D capturing device is continuously tracked/detected in step S7.

For example, if the surgeon determines that re-registration is required, the second registration can be repeated at any time, in particular if the tissue has moved. If the condition B1 of a new registration is fulfilled (Yes), the registering method proceeds again to step S5 and a correlation with the 3D image data 3DA is determined on the basis of the three-dimensional intracorporeal image IA of the 3D capturing device.

If no new registration is desired (No), the method advances to condition B2. As long as condition B2 does not specify that the method should be terminated, step S7 is executed again. Otherwise the method is terminated.

Although the present disclosure is set forth in particular in the context of neurosurgery, it can generally be applied in other regions such as spinal intervention, ENT surgery as well as general surgery.

1.-10. (canceled)

11. A surgical assistance system for use in a surgical intervention, the surgical assistance system comprising:
at least one imaging 3D capturing device adapted to create a three-dimensional intracorporeal image for a patient and provide the three-dimensional intracorporeal image in a computer-readable manner;
a tracking system adapted to detect and track at least one surgical intervention region of the patient;
a data provision unit adapted to provide digital 3D image data of the patient; and
a control unit adapted to process the three-dimensional intracorporeal image, data of the tracking system and the digital 3D image data of the patient,
the surgical assistance system being adapted to register the digital 3D image data of the patient on an outer surface of the patient as a first registration, and
the control unit being adapted to determine at least a landmark and/or a surface and/or a three-dimensional volume of an intracorporeal structure as an intracorporeal reference from the three-dimensional intracorporeal image and/or the digital 3D image data, and based on the first registration and based on the intracorporeal reference, to register the digital 3D image data to the intracorporeal structure as a second registration to increase accuracy of registration, and
the control unit being adapted to perform the second registration based on a rigid transformation.

12. The surgical assistance system according to claim 11, further comprising at least one of:
a surgical 3D microscope;
a surgical 3D endoscope; and
a medical instrument with an optical 3D camera as a 3D capturing device for creating the three-dimensional intracorporeal image.

13. The surgical assistance system according to claim 11, wherein the control unit is adapted to determine at least one point-like landmark and/or at least one surface for the second registration in the three-dimensional intracorporeal image.

14. The surgical assistance system according to claim 11, wherein the control unit is adapted to detect exactly one single landmark and to perform a registration based on said one single landmark with only one transformation correction with respect to a translation.

15. The surgical assistance system according to claim 11, wherein the tracking system comprises at least one of:
an infrared-based navigation system;
an electromagnetic navigation system; and
an image-processing navigation system.

16. The surgical assistance system according to claim 11, wherein the control unit is adapted to perform the first registration via a point-to-point matching and/or a surface matching using an outer surface of the patient.

17. The surgical assistance system according to claim 11, wherein the control unit is adapted to perform the first registration via the tracking system on an external surface of the patient.

18. The surgical assistance system according to claim 11, wherein the control unit is adapted to perform the first registration via an external 3D stereo camera of the tracking system on a face of the patient.

19. The surgical assistance system according to claim 11, wherein the control unit is adapted to perform the second registration a plurality of times intraoperatively.

20. A registering method for registering 3D image data on a tissue of a patient, the registering method comprising the steps of:
detecting 3D image data of a patient;
a first registration step, in which the 3D image data is registered onto an outer surface of the patient as a first registration;
creating a three-dimensional intracorporeal image of the patient by a 3D capturing device;
determining at least a landmark and/or a surface and/or a volume in the three-dimensional intracorporeal image and further in the 3D image data as an intracorporeal reference; and
a second registration step, in which the 3D image data is registered to an intracorporeal structure of the patient captured as a three-dimensional intracorporeal image as a second registration, based on the first registration and the determined intracorporeal reference, in order to increase the accuracy of registration,
wherein the second registration step comprises performing a rigid transformation in order to register the 3D image data to the intracorporeal structure.

21. The registering method according to claim 20, wherein the second registration step comprises detecting at least a landmark and/or a surface in the three-dimensional intracorporeal image.

22. The registering method according to claim 20, wherein the first registration step is performed preoperatively and the second registration step is performed intraoperatively.

23. The registering method according to claim 20, wherein the first registration step is carried out by an external 3D stereo camera of the tracking system on a face of the patient.

24. The registering method according to claim 20, wherein, after the second registration step, the registering method further comprises repeating the second registration step.

25. The registering method according to claim 20, wherein, in the second registration step, a second transformation matrix is determined, which performs a transformation only within defined limits with respect to a first transformation matrix of the first registration step.

26. A computer-readable storage medium comprising commands which, when executed by a computer, cause the computer to execute the registering method according to claim 20.
